(19) United States
(12) Patent Application Publication
(10) Pub. No.: US 2008/0119527 A1
(43) Pub. Date: May 22, 2008

(54) Composition for combating the localized hyperpigmentation of dark skin

(75) Inventor: Francine Baldo, Sceaux (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 11/898,426

(22) Filed: Sep. 12, 2007

Related U.S. Application Data
(60) Provisional application No. 60/847,918, filed on Sep. 29, 2006.

(30) Foreign Application Priority Data
Sep. 15, 2006 (FR) 06 53752

(51) Int. Cl. A61K 31/4415; A61K 31/4025; A61K 31/20; A61P 17/00

(52) U.S. Cl. 514/355; 514/422; 514/558

(57) ABSTRACT

The present invention relates to a composition comprising, in a physiologically acceptable medium, at least one copolymer of a styrene monomer and of an ethylenically unsaturated dicarboxylic acid and at least one anti-seborrhoeic active agent.

This non provisional application claims the benefit of French Application No. 06 53752 filed on Sep. 15, 2006 and U.S. Provisional Application No. 60/847,918 filed on Sep. 29, 2006.

The present invention relates to a cosmetic composition for correcting or preventing disorders associated with greasiness of a dark skin, especially by the action of reducing the secretion of sebum while at the same time imparting depigmenting properties, comprising at least one copolymer of a styrene monomer and of an ethylenically unsaturated dicarboxylic acid and at least one anti-seborrhoeic active agent. The invention also relates to a cosmetic process for correcting or preventing skin disorders associated with hyperseborrhoea of dark skin. More particularly, this process makes it possible to combat both hyperseborrhoea and localized hyperpigmentation that may occur at the site of cicatrization of acne lesions on dark skin.

In general, three main skin types may be distinguished: dry skin, greasy skin and combination skin.

Greasy skin is hyperseborrhoeic and characterized by an exaggerated secretion and excretion of sebum. Conventionally, a sebum level of greater than 200 μg/cm² on the forehead is considered as being characteristic of greasy skin. For the purposes of the present invention, greasy skin is skin of shiny, thick appearance whose follicular orifices are dilated or filled with tiny horny spicules, or even with comedons (however, this results more from a phenomenon of retention than from an increase in excretion). Greasy skin is often associated with a desquamation defect, and a thick skin grain. Another cutaneous sign of greasy skin is the presence of lesions.

In addition to these aesthetic disorders, the excess sebum may serve as a support for the anarchic growth of saprophytic bacterial flora (in particular Propionibacterium acnes and Pityrosporum ovale), and cause comedons and/or acne lesions.

Acne lesions appear in the form of spots that end by drying up.

In the case of dark skin, especially of phototype IV to VI, the formation of the acne lesions and then of the spots mentioned above often causes scars to appear, which are generally accompanied by a hyperpigmentation of the skin in the area of cicatrization, which further accentuates the cutaneous defect emanating from the acne lesion itself.

This may in particular be observed for dark skin of African type. These localized hyperpigmentation areas appear in the form of darker marks that then take a long time to disappear naturally. The disappearance process may take up to several weeks. One solution for treating these marks is to use a depigmenting active agent. However, standard depigmenting active agents such as hydroquinone and derivatives thereof, or resorcinol, are too aggressive and are unsuitable for treating acne. The reason for this is that these aggressive active agents accentuate the irritation and inflammation produced by the acne, and their use is thus incompatible with an anti-acne treatment.

There is thus a need for a cosmetic composition for overcoming the localized hyperpigmentations described above encountered in the case of dark skin, while at the same time combating greasy skin and/or hyperseborrhoea and preventing the acne lesions resulting therefrom.

The copolymer of a styrene monomer and of an ethylenically unsaturated dicarboxylic acid has already been used in cosmetic compositions.

In particular, it is known from document EP 1 374 852 to combine it with an oxidation-sensitive active agent for the purpose of stabilizing the said active agent.

Document WO 2004/028 483 moreover discloses an aqueous composition comprising at least one metal salt of phosphorylated ascorbic acid, at least one water-soluble UV-screening agent comprising at least one sulfonic function and at least one maleic anhydride polymer, copolymers comprising maleic anhydride monomers and comonomers of styrene type being described.

Neither of these documents mentions the beneficial effect of such copolymers in compositions intended for treating greasy skin and/or hyperseborrhoea and/or for preventing acne lesions and also the imperfections resulting therefrom.

The inventors have discovered, surprisingly, that the combination of at least one copolymer of a styrene monomer and of an ethylenically unsaturated dicarboxylic acid and of at least one anti-seborrhoeic active agent has advantageous activity for treating greasy skin and/or hyperseborrhoea and for preventing the acne lesions that result therefrom and more particularly for avoiding aesthetic disorders associated with the overproduction of sebum, in particular the scars that appear more particularly in the case of dark skin. The reason for this is that the excess sebum may induce, as outlined previously, the anarchic growth of certain pathogenic bacteria such as Propionibacterium acnes, and lead to acne lesions. These acne lesions finish by drying up and a scar then appears, which, for example, in the case of dark skin, may lead to inaesthetic hyperpigmentation at the site of cicatrization. The discovered combination makes it possible both to combat hyperseborrhoea and to reduce the local browning formed during the cicatrization of the acne lesions.

More specifically, one subject of the invention is thus a composition comprising, in a physiologically acceptable medium, at least one copolymer of a styrene monomer and of an ethylenically unsaturated dicarboxylic acid and at least one anti-seborrhoeic active agent.

A subject of the invention is also a cosmetic process for treating greasy skin and/or hyperseborrhoea and for preventing the acne lesions resulting therefrom and/or for combating aesthetic disorders or imperfections associated with the overproduction of sebum on dark skin types, especially of phototype IV to VI, comprising at least one step that consists in applying to the skin a composition according to the invention.

A subject of the invention is also a cosmetic process for combating both greasy skin and hyperpigmentation localized at the sites of cicatrization of acne lesions resulting from the hyperseborrhoea of dark skin types, especially of phototype IV to VI, comprising at least one step that consists in applying to the skin a composition according to the invention.

A dermatological method for treating greasy skin and/or the hyperseborrhoea of dark skin types comprising the topical application to zones of the skin of a person in need thereof of an effective amount of a composition comprising a combination of at least one copolymer of a styrene monomer and of an ethylenically unsaturated dicarboxylic acid and of at least one anti-seborrhoeic active agent.

In particular, a dark skin targeted by the invention belongs to the phototypes IV to VI of the Fitzpatrick classification.

For the purposes of the present invention, the term “dark skin” means in particular skin whose mean lightness L*, measured on the forehead, the cheekbones and the chin in the CIE 1976 colorimetric space, is less than 55. The saturation C* may be, for example, between 10 and 30 and especially between 12 and 28. The hue angle values h in degrees may be, for example, between about 38° and about 54°. The lightness values L* may be less than or equal to 50, or even 45 or 40 for darker skin types, while at the same time remaining, for the majority of skin types, greater than 30.

These skin types may also be classified on the basis of their reactivity to the effects of solar radiation according to the scale proposed by Fitzpatrick.

According to this scale, the various existing skin types may be distinguished according to the following phototypes:

| Type | Skin reactivity | Origin |
|---|---|---|
| I | Always burns, never tans | Celtic |
| II | Always burns, tans very little | Germanic |
| III | Burns moderately, tans gradually | European |
| IV | Burns lightly, tans very easily | Mediterranean, Indian, |
| | | Asian |
| V | Rarely burns, tans deeply | Middle Eastern - South |
| | | American |
| VI | Never burns, highly pigmented | African |

The dark skin types that are more particularly the subject of the present invention belong to phototypes IV to VI.

Populations of ethnic origin, for instance African, Maghrebian, Indian and Afro-American are more particularly representative of phototypes IV, V and VI.

Copolymer of a Styrene Monomer and of an Ethylenically Unsaturated Dicarboxylic Acid

The copolymer used in the composition according to the invention is a copolymer of a styrene monomer and of an ethylenically unsaturated dicarboxylic acid.

Styrene monomers that may be mentioned include styrene and α-methylstyrene, preferably styrene.

Ethylenically unsaturated dicarboxylic acid monomers that may be mentioned include maleic acid, itaconic acid and anhydride derivatives thereof hydrolysed after polymerization and especially maleic anhydride and itaconic anhydride.

Copolymers that are more particularly suitable for use in the invention are copolymers obtained by copolymerization of one or more maleic or itaconic anhydride unit(s) followed by hydrolysis thereof.

The maleic anhydride or itaconic anhydride units may be partially or totally hydrolysed.

The said copolymer is preferably in salt form, especially in the form of alkaline salts, for example in the form of ammonium, sodium, potassium or lithium salts, and preferably in the form of the sodium salt.

In one advantageous aspect of the invention, the copolymer has a mole fraction of ethylenically unsaturated dicarboxylic acid units of between 0.1 and 0.9, preferably between 0.4 and 0.9 and more preferentially between 0.4 and 0.6.

Preferably, the said polymer has a solubility in water at 25° C. of greater than or equal to 2 g/l.

The weight-average molar mass of the styrene/ethylenically unsaturated dicarboxylic acid copolymer is preferably between 1000 and 500 000 and preferably between 1000 and 50 000.

Preferentially, a copolymer of styrene and of maleic acid (or of hydrolysed maleic anhydride), in a styrene/maleic acid mole ratio ranging from 40/60 to 60/40, in particular equal to 50/50, will be used.

In the context of the present invention, the copolymer is advantageously a copolymer of styrene and of maleic acid.

It is possible to use, for example, the styrene/maleic anhydride (50/50) copolymer, hydrolysed in the form of the ammonium salt at 30% in water, sold under the reference SMA1000H® by the company Cray Valley or the styrene/maleic anhydride copolymer (50/50) hydrolysed in the form of the sodium salt at 40% in water, sold under the reference SMA1000HNa® by the company Cray Valley (and having the INCI name: sodium styrene/MA copolymer).

Certain polymers of styrene and of an ethylenic dicarboxylic acid show good skin depigmenting and antipigmenting activity. The use of these polymers also has the advantage of being non-irritant, non-toxic and non-allergenic to the skin. Examples 1 and 2 given below more particularly illustrate this property.

The copolymer is present in the composition used according to the invention in an amount sufficient to give it a skin-depigmenting property. Preferably, the copolymer is present in a content ranging from 0.1% to 40% by weight relative to the total weight of the composition, more particularly in a content ranging from 0.1% to 20% by weight and preferentially ranging from 0.1% to 10% by weight relative to the total weight of the composition.

According to one particular embodiment of the invention, the composition is free of an oxidation-sensitive hydrophilic active ingredient.

The term “hydrophilic active agent” means a compound whose solubility in water is less than 0.25% at room temperature (25° C.).

The term “oxidation-sensitive hydrophilic active agent” means any hydrophilic active agent of natural or synthetic origin capable of undergoing degradation via an oxidation mechanism. This oxidation phenomenon may have several causes, in particular the presence of oxygen, of light, of metal ions, a high temperature, or even certain pH conditions.

In particular, the oxidation-sensitive hydrophilic active agent may be ascorbic acid or a derivative thereof chosen especially from 5,6-di-O-dimethylsilylascorbate, the potassium salt of dl-α-tocopheryl-dl-ascorbyl phosphate, magnesium ascorbyl phosphate, sodium ascorbyl phosphate, ascorbyl glucoside; phloroglucinol and kojic acid.

Anti-Seborrhoeic Active Agent

The composition according to the present invention comprises at least one anti-seborrhoeic active agent. The term “anti-seborrhoeic active agent” means a compound capable of regulating the activity of the sebaceous glands.

As anti-seborrhoeic agents that may be used in the composition according to the invention, mention may be made of:
retinoic acid, benzoyl peroxide, sulfur, vitamin B6 (or pyridoxine), selenium chloride and sea fennel;
mixtures of extract of cinnamon, of tea and of octanoylglycine such as Sepicontrol A5 TEA® from SEPPIC;
the mixture of cinnamon, sarcosine and octanoylglycine sold especially by the company SEPPIC under the trade name Sepicontrol A5®;
zinc salts such as zinc gluconate, zinc pyrrolidonecarboxylate (or zinc pidolate), zinc lactate, zinc aspartate, zinc carboxylate, zinc salicylate and zinc cysteate;
copper derivatives and in particular copper pidolate such as Cuivridone® from Solabia;
extracts of plants of the species Arnica montana, Cinchona succirubra, Eugenia caryophyllata, Humulus lupulus, Hypericum perforatum, Mentha piperita, Rosmarinus officinalis, Salvia oficinalis and Thymus vulgaris, all sold, for example, by the company Maruzen;
extracts of meadowsweet (Spiraea ulmaria), such as the product sold under the name Sebonormine® by the company Silab;
extracts of the alga Laminaria saccharina, such as the product sold under the name Phlorogine® by the company Biotechmarine;
mixtures of extracts of salad burnet root (Sanguisorba officinalis/Poterium officinale), of ginger rhizomes (Zingiber officinalis) and of cinnamon bark (Cinnamomum cassia), such as the product sold under the name Sebustop® by the company Solabia;
linseed extracts, such as the product sold under the name Linumine® by the company Lucas Meyer;
Phellodendron extracts, such as those sold under the name Phellodendron extract BG by the company Maruzen or Oubaku liquid B by the company Ichimaru Pharcos;
mixtures of argan oil, of Serenoa serrulata (saw palmetto) extract and of sesame seed extract, such as the product sold under the name Regu SEB® by the company Pentapharm;
mixtures of extracts of willowherb, of Terminalia chebula, of nasturtium and of bioavailable zinc (microalgae), such as the product sold under the name Seborilys® by the company Green Tech;
extracts of Pygeum afrianum, such as the product sold under the name Pygeum afrianum sterolic lipid extract by the company Euromed;
extracts of Serenoa serrulata, such as the products sold under the name Viapure Sabal by the company Actives International or those sold by the company Euromed;
mixtures of extracts of plantain, of Berberis aquifolium and of sodium salicylate, such as the product sold under the name Seboclear® by the company Rahn;
clove extract, such as the product sold under the name Clove extract powder by the company Maruzen;
argan oil, such as the product sold under the name Lipofructyl® by Laboratoires Sérobiologiques;
lactic protein filtrates, such as the product sold under the name Normaseb® by the company Sederma;
extracts of the alga Laminaria, such as the product sold under the name Laminarghane® by the company Biotechmarine;
oligosaccharides of the alga Laminaria digitata, such as the product sold under the name Phycosaccharide AC by the company Codif;
sugar cane extracts, such as the product sold under the name Policosonol® by the company Sabinsa;
sulfonated shale oil, such as the product sold under the name Ichthyol Pale® by the company Ichthyol;
European meadowsweet (Spiraea ulmaria) extracts, such as the product sold under the name Cytobiol® Ulmaire by the company Libiol;
sebacic acid, especially sold in the form of a sodium polyacrylate gel under the name Sebosoft® by the company Sederma;
glucomannans extracted from konjac tuber and modified with alkylsulfonate chains, such as the product sold under the name Biopol Beta by the company Arch Chemical;
extracts of Sophora angustifolia, such as those sold under the name Sophora powder or Sophora extract by the company Bioland;
extracts of Cinchona succirubra bark, such as the product sold under the name Red Bark HS by the company Alban Muller;
extracts of Quillaja saponaria, such as the product sold under the name Panama wood HS by the company Alban Muller;
glycine grafted onto an undecylenic chain, such as the product sold under the name Lipacide UG OR by the company SEPPIC;
the mixture of oleanolic acid and of nordihydroguaiaretic acid, such as the product sold in the form of a gel under the name AC.Net by the company Sederma;
phthalimidoperoxyhexanoic acid;
tri(C₁₂-C₁₃)alkyl citrate sold under the name Cosmacol® ECI by the company Sasol; tri(C₁₄-C₁₅)alkyl citrate sold under the name Cosmacol® ECL by the company Sasol;
10-hydroxydecanoic acid, and especially mixtures of 10-hydroxydecanoic acid, of sebacic acid and of 1,10-decanediol, such as the product sold under the name Acnacidol® BG by the company Vincience; and
mixtures thereof.

Preferred anti-seborrhoeic active agents that may be mentioned include:
benzoyl peroxide and vitamin B6 (or pyridoxine),
zinc salts such as zinc gluconate, zinc pyrrolidone carboxylate (or zinc pidolate), zinc lactate, zinc aspartate, zinc carboxylate, zinc salicylate and zinc cysteate;
meadowsweet (Spiraea ulmaria) extracts, such as the product sold under the name Sebonormine® by the company Silab;
extracts of the alga Laminaria saccharina, such as the product sold under the name Phlorogine® by the company Biotechmarine;
mixtures of extracts of salad burnet root (Sanguisorba officinalis/Poterium officinale), of ginger rhizomes (Zingiber officinalis) and of cinnamon bark (Cinnamomum cassia), such as the product sold under the name Sebustop® by the company Solabia;
clove extract, such as the product sold under the name Clove extract powder by the company Maruzen;
lactic protein filtrates, such as the product sold under the name Normaseb® by the company Sederma;
European meadowsweet (Spiraea ulmaria) extracts, such as the product sold under the name Cytobiol® Ulmaire by the company Libiol;
sebacic acid, especially sold in the form of a sodium polyacrylate gel under the name Sebosoft® by the company Sederma;
glycine grafted onto an undecylenic chain, such as the product sold under the name Lipacide UG OR by the company SEPPIC;
tri(C₁₂-C₁₃)alkyl citrate sold under the name Cosmacol® ECI by the company Sasol; tri(C₁₄-C₁₅)alkyl citrate sold under the name Cosmacol® ECL by the company Sasol;
10-hydroxydecanoic acid, and especially mixtures of 10-hydroxydecanoic acid, of sebacic acid and of 1,10-decanediol, such as the product sold under the name Acnacidol® BG by the company Vincience; and
mixtures thereof.

Preferentially, the anti-seborrhoeic active agent is chosen from:
zinc salts such as zinc gluconate, zinc pyrrolidone carboxylate (or zinc pidolate), zinc lactate, zinc aspartate, zinc carboxylate, zinc salicylate and zinc cysteate; and preferably zinc pyrrolidonecarboxylate (or zinc pidolate) or zinc salicylate;
clove extract, such as the product sold under the name Clove extract powder by the company Maruzen;
glycine grafted onto an undecylenic chain, such as the product sold under the name Lipacide UG OR by the company SEPPIC;
tri(C₁₂-C₁₃)alkyl citrate sold under the name Cosmacol® ECI by the company Sasol; tri(C₁₄-C₁₅)alkyl citrate sold under the name Cosmacol® ECL by the company Sasol;
and mixtures thereof.

The anti-seborrhoeic active agent is, for example, present in a content ranging from 0.1% to 10% by weight, preferably from 0.1% to 5% by weight and preferentially from 0.5% to 3% by weight relative to the total weight of the composition.

Anti-Acne Active Agent

In one advantageous aspect of the invention, the composition may also comprise at least one anti-acne active agent.

The term “anti-acne active agent” especially means any active agent that has effects on the specific flora of greasy skin, for instance Propionibacterium acnes (P. acnes). These effects may be bactericidal.

Antibactericidal active agents that may especially be mentioned include:
active agents and preserving agents with antimicrobial activity mentioned in patent application DE 103 24 567, which is incorporated into the present invention by reference,
Asiatic acid,
the monoethanolamine salt of 1-hydroxy-4-methyl 6-trimethylpentyl-2-pyridone (INCI name: piroctone olamine), sold especially under the name Octopirox® by the company Clariant;
citronellic acid, perillic acid (or 4-isopropenylcyclohex-1-enecarboxylic acid),
glyceryl 2-ethylhexyl ether (INCI name: ethylhexylglycerine), for example sold under the name Sensiva SC 50 by the company Shulke & Mayr,
glyceryl caprylate/caprate, for example sold under the name Capmul MCM® by the company Abitec;
sodium calcium phosphosilicate, especially sold under the names Bioactive Glasspowder® and Actysse Premier BG® by the company Schott Glass;
silver-based particles, for example those sold under the name Métashine ME 2025 PS® by the company Nippon Sheet Glass;
hop cone extract (Humulus lupulus) obtained by supercritical CO₂ extraction, such as the product sold under the name HOP CO2-TO Extract® by the company Flavex Naturextrakte,
St.-John's Wort extract obtained by supercritical CO₂ extraction, such as the product sold under the name St.-John's Wort CO2-TO Extract® by the company Flavex Naturextrakte,
the mixture of extracts of roots of Scutellaria baicalensis, of Paeonia suffruticosa and Glycyrrhiza glabra, such as the product sold under the name BMB-CF® by the company Naturogin,
argan tree extract, for instance Argapure LS9710® from Cognis;
bearberry leaf extracts, for instance the product sold under the name Melfade-J by the company Pentapharm;
10-hydroxy-2-decanoic acid such as Acnacidol P® from Vincience, sodium ursolate, azelaic acid, diiodomethyl p-tolyl sulfone such as Amical Flowable® from Angus, malachite powder, zinc oxide such as Zincare® from Elementis GMBH, octadecenedioic acid such as Arlatone dioic DCA® from Uniqema; ellagic acid; 2,4,4′-trichloro-2′-hydroxydiphenyl ether (or triclosan), 1-(3′,4′-dichlorophenyl)-3-(4′-chlorophenyl)urea (or triclocarban), 3,4,4′-trichlorocarbanilide, 3′,4′,5′-trichlorosalicylanilide, phenoxyethanol, phenoxypropanol, phenoxyisopropanol, hexamidine isethionate, metronidazole and salts thereof, miconazole and salts thereof, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and salts thereof, benzoyl peroxide, 3-hydroxybenzoic acid, 4-hydroxybenzoic acid, phytic acid, N-acetyl-L-cysteine, lipoic acid, azelaic acid and salts thereof, arachidonic acid, resorcinol, 3,4,4′-trichlorocarbanalide, octoxyglycerine or octoglycerine, octanoylglycine such as Lipacid C8G® from SEPPIC, caprylyl glycol, 10-hydroxy-2-decanoic acid, dichlorophenylimidazoldioxolane and derivatives thereof described in patent application WO 93/18743, iodopropynyl butylcarbamate, 3,7,11-trimethyldodeca-2,5,10-trienol or farnesol, phytosphingosines; quaternary ammonium salts, for instance cetyltrimethylammonium salts and cetylpyridinium salts, and
mixtures thereof.

Mention may also be made of certain surfactants with an antimicrobial effect, for instance sodium cocoamphoacetate or disodium diacetate such as Miranol C2M Conc. NP, betaines, for instance the cocoyl betaine Genagen KB from Clariant, sodium lauryl ether sulfate, for instance Emal 270 D from Kao, decyl glucoside, for instance Plantacare 2000 UP, branched C₁₂₋₁₃ dialkyl malates, for instance Cosmacol EMI, propylene glycol monoesters, for instance propylene glycol monolaurate, monocaprylate or monocaprate, lauryldimethylamine betaine, for instance Empigen BB/LS, and also polyquaternary ammoniums such as Quatemium-24 or Bardac 2050 from Lonza and those described in patent FR 0 108 283, and mixtures thereof.

As preferred antimicrobial agents, an agent chosen from caprylyl glycol, octoglycerine or octoxyglycerine, and 10-hydroxy-2-decanoic acid, and mixtures thereof, will be used in the compositions of the invention.

Other additional anti-acne active agents may be added to the above-mentioned anti-acne active agents. Mention may be made especially of active agents with bacterial anti-adhesion effects or agents that act on the biofilm of bacteria to prevent them from multiplying.

As agents for preventing and/or reducing the adhesion of micoorganisms, mention may be made especially of: phytanetriol and derivatives thereof as described in patent application EP 1 529 523, plant oils such as wheatgerm oil, calendula oil, castor oil, olive oil, avocado oil, sweet almond oil, groundnut oil, jojoba oil, sesame seed oil, apricot kernel oil, sunflower oil and macadamia oil, described in patent EP 1 133 979, or certain surfactants such as disodium cocoamphodiacetate, oxyethylenated (7 EO) glyceryl cocoate, 18-hexadecenyl succinate, octoxyglyceryl palmitate, octoxyglyceryl behenate, dioctyl adipate, PPG-15 stearyl ether, and the branched C₁₂-C₁₃ dialkyl tartrates described in patent EP 1 129 694, and mixtures thereof.

In particular with regard to the propagation of P. acnes, or as active agents that act on the biofilm of bacteria to prevent them from proliferating, mention may be made of pentylene glycol, Nylon-66 (polyamide 66 fibres), rice bran oil, polyvinyl alcohol such as Celvol 540 PV Alcohol® from Celanese Chemical, rapeseed oil such as Akorex L® from Karlshamns, and fructose derivatives, and mixtures thereof.

The anti-acne active agent may be present in a content ranging from 0.01% to 10% by weight and preferably from 0.05% to 5% by weight relative to the total weight of the composition.

Desquamating Agent

In another advantageous aspect of the invention, the compositions according to the present invention may also comprise a desquamating agent.

The term “desquamating agent” means any compound capable of acting:
either directing on desquamation by promoting exfoliation, such as β-hydroxy acids, in particular salicylic acid and derivatives thereof (including 5-n-octanoylsalicylic acid); α-hydroxy acids, such as glycolic acid, citric acid, lactic acid, tartaric acid, malic acid or mandelic acid; 8-hexadecene-1,16-dicarboxylic acid; gentisic acid and derivatives thereof; oligofucoses; cinnamic acid; Saphora japonica extract; resveratrol, and certain jasmonic acid derivatives;
or on the enzymes involved in the desquamation or degradation of comeodesmosomes, glycosidases, stratum corneum chymotryptic enzyme (SCCE) or other proteases (trypsin, chymotrypsin-like). Mention may be made of the aminosulfonic compounds as described in patent application W 0.2/39975 and in particular (N-2-hydroxyethylpiperazine-N-2-ethane)sulfonic acid (HEPES); 2-oxothiazolidine-4-carboxylic acid (procysteine) derivatives; derivatives of α-amino acids of glycine type (as described in EP-0 852 949, and also sodium methyl glycine diacetate sold by BASF under the trade name Trilon M); honey; O-octanoyl-6-D-maltose and N-acetylglucosamine.

The abovementioned mixtures of desquamating agents are also included as desquamating agents.

As other desquamating agents that may be used in the composition according to the invention, mention may be made of:
oligofructoses, EDTA and derivatives thereof, laminaria extracts, o-linoleyl-6D-glucose, (3-hydroxy-2-pentylcyclopentyl)acetic acid, glycerol trilactate, O-octanyl-6′-D-maltose, S-carboxymethylcysteine, silicious derivatives of salicylate such as those described in patent EP 0 796 861, oligofucases such as those described in patent EP 0 218 200, jasmonic acid and derivatives such as those described in patent applications EP 1 333 022 and EP 1 333 021,
extract of the flowers of ficus Opuntia indica (Exfolactive® from Silab),
mixtures thereof.

Preferred desquamating agents that may be mentioned include β-hydroxy acids such as 5-n-octanoyl salicylic acid; glycolic acid, citric acid, lactic acid, tartaric acid, malic acid or mandelic acid; (N-2 hydroxyethylpiperazine-N-2-ethane)sulfonic acid (HEPES); extract of Saphora japonica; honey; N-acetyl glucosamine; sodium methyl glycine diacetate, and mixtures thereof.

Even more preferentially, a desquamating agent chosen from 5-n-octanoyl salicylic acid; urea; (N-2-hydroxyethylpiperazine-N-2-ethane)sulfonic acid (HEPES); extract of Saphora japonica; honey; N-acetyl glucosamine; sodium methyl glycine diacetate, and mixtures thereof, will be used in the compositions of the invention.

Other Agents

According to yet another advantageous aspect of the invention, the compositions according to the present invention may also comprise at least one calmative and/or at least one astringent active agent and/or at least one matting active agent and/or at least one cicatrizing active agent and/or at least one active agent acting on keratinocyte proliferation.

The term “calmative” means a compound that can reduce the sensation of stinging, itching or tautness of the skin.

As calmatives that may be used in the composition according to the invention, mention may be made of: procyannidol oligomers, vitamins E, C B5 and B3, caffeine and derivatives thereof, pentacylic triterpenes and plant extracts containing them, β-glycyrrhetinic acid and salts or derivatives thereof (stearyl glycyrrhetate, 3-stearoyloxyglycyrrhetic acid or glycyrrhetinic acid monoglucuronide) and also plants containing them (e.g.: Glycyrrhiza glabra), oleanolic acid and salts thereof, ursolic acid and salts thereof, boswellic acid and salts thereof, betulinic acid and salts thereof, an extract of Paeonia suffruticosa and/or lactiflora, an extract of Laminaria saccharina, extracts of Centella asiatica, Canola oil, bisabolol, the phosphoric diester of vitamin E and C, for instance Sepivital EPC® from SEPPIC, camomile extracts, allantoin, omega-3 unsaturated oils such as musk rose oil, blackcurrant oil, Ecchium oil, fish oil or beauty-leaf oil, plankton extracts, capryloyl glycine, a mixture of water lily blossom extract and of palmitoylproline, such as the product sold under the name Seppicalm VG® by the company SEPPIC, an extract of Boswellia serrata, an extract of Centipeda cunninghami, such as the product sold under the name Cehami PF® by the company TRI-K Industries, an extract of sunflower seeds, in particular Hélioxine® from Silab, an extract of Linum usitatissimum seeds, for instance Sensiline® from Silab, tocotrienols, piperonal, an extract of Epilobium angustifolium, such as the product sold under the name Canadian Willowherb Extract by the company Fytokem Products, Aloe vera, phytosterols, cornflower water, rose water, an extract of mint, in particular of mint leaves, for instance Calmiskin® from Silab, aniseed derivatives, filamentous bacteria, for instance Vitreoscilla fliformis as described in patent EP 761 204, an extract of rose petals, for instance Rose Flower Herbasol® extract from the company Cosmetochem, shea butter, a mixture of the waxy fraction of barley seeds obtained by supercritical CO₂, of shea butter and of argan oil, for instance Stimu-tex AS® from Pentapharm, alkaline-earth metal salts, especially of strontium, and mixtures thereof.

As preferred calmatives according to the invention, use will be made of:
β-glycyrrhetinic acid and salts or derivatives thereof (stearylglycyrrhetate, 3-stearoyloxyglycyrrhetic acid or glycyrrhetinic acid monoglucuronide) and also plants containing them (e.g. Glycyrrhiza glabra); ursolic acid and salts thereof; extracts of Centella asiatica, Canola oil, bisabolol; camomile extracts, allantoin; a mixture of extract of water lily blossom and of palmitoylproline, such as the product sold under the name Seppicalm VG® by the company SEPPIC; Aloe vera, rose water, extract of mint, in particular of mint leaves, such as Calmiskin® from Silab, filamentous bacteria such as Vitreoscilla filiformis as described in patent EP 761 204, an extract of rose petals such as Rose Flower Herbasol® extract from the company Cosmetochem, and shea butter, and mixtures thereof.

Preferentially, the calmative is chosen from:
bisabolol; a mixture of extracts of water lily blossom and of palmitoylproline, such as the product sold under the name Seppicalm VG® by the company SEPPIC; Aloe vera, rose water, an extract of mint, in particular of mint leaves such as Calmiskin® from Silab, filamentous bacteria such as Vitreoscilla filiformis as described in patent EP 761 204, an extract of rose petals such as Rose Flower Herbasol® extract from the company Cosmetochem, and shea butter, and mixtures thereof.

According to the invention, the term “astringents” means agents for combating the dilation of the sebaceous follicles.

As astringents that may be used in the composition according to the invention, mention may be made of extracts of mushroom pulp (polyporus officinalis), for instance Laricyl LS8865® from Cognis, extracts of Terminalia catappa and sambucus nigra, for instance Phytofirm LS9120® from Cognis, extracts of gall nut, for instance Tanlex VE® from Ichimaru Pharcos, aluminium hydroxychloride, centella extracts (e.g. Plantactiv centella from Cognis), dicetyl dimethylammonium chloride, for instance Varisoft 432 CG® from Degussa, common horsechestnut extracts, mallow extracts, witch-hazel extracts, sweet almond extracts, marshmallow root extracts and linseed extracts, for instance Almondermin LS 3380® from Cognis, burdock extracts, nettle extracts, birch extracts, horsetail extracts, camomile extracts, for instance those sold under the name Extrapone 9 Special® by the company Symrise, scullcap extracts, European meadowsweet extracts (for example Cytobiol Ulmaire from Libiol), a mixture of extracts of white ginger, of horsetail, of nettle, of rosemary and of yucca, for instance Herb extract B1348® from Bell Flavors & Fragrances, extracts of acacia, of elm, of white willow, of cinnamon, of birch and of meadowsweet, Panama sapogenins, zinc phenolsulfonate from Interchemical, extracts of gentiane, of cucumber and of walnut, the mixture of extracts of Ratanhia, of grapefruit, of gumweed and of oak gall, for instance Epilami® from Alban Muller.

As preferred astringents according to the invention, use will be made of scullcap extracts, European meadowsweet extracts, meadowsweet extracts, gentiane extracts and burdock extracts, and mixtures thereof.

Examples of cicatrizing agents that may especially be mentioned include:
allantoin, urea, certain amino acids, for instance hydroxyproline, arginine, and serine, and also extracts of white lily (for instance Phytélène Lys 37EG 16295 from Indena), a yeast extract, for instance the cicatrizing agent LS LO/7225B from Laboratoires Sériobiologiques), tamanu oil, extract of Saccharomyces cerevisiae, for instance Biodynes®V TRF® from Arch Chemical, oat extracts, chitosan and derivatives, for instance chitosan glutamate, carrot extracts, artemia extract, for instance GP4G® from Vincience, sodium acexamate, lavandin extracts, propolis extracts, ximeninic acid and salts thereof, rose hip oil, marigold extracts, for instance Souci Ami® Liposolible from Alban Muller, horsetail extracts, lemon peel extracts, for instance Herbasol® citron from Cosmetochem, helichrysum extracts and common yarrow extracts.

As preferred cicatrizing agents according to the invention, use will be made of tamanu oil, sodium acexamate, horsetail extracts and helichrysum extracts, and mixtures thereof.

The term “matting agent” means agents intended to make the skin visibly more matt and less shiny.

The matting effect of the agent and/or composition containing it may especially be evaluated using a gonioreflectometer, by measuring the ratio R between the specular reflection and the scattered reflection. A value of R of less than or equal to 2 generally reflects a matting effect.

The matting agent may especially be chosen from a rice starch or a corn starch, kaolinite, talc, a pumpkin seed extract, cellulose microbeads, plant fibres, synthetic fibres, in particular polyamide fibres, expanded acrylic copolymer microspheres, polyamide powders, silica powders, polytetrafluoroethylene powders, silicone resin powders, acrylic polymer powders, wax powders, polyethylene powders, powders of elastomeric crosslinked organopolysiloxane coated with silicone resin, talc/titanium dioxide/alumina/silica composite powders, amorphous mixed silicate powders, silicate particles and especially mixed silicate particles, and mixtures thereof.

Examples of matting agents that may especially be mentioned include:
rice or corn starch, in particular an aluminium starch octenyl succinate sold under the name Dry Flo® by the company National Starch;
kaolinite;
silicas;
talc;
a pumpkin seed extract as sold under the name Curbilene® by the company Indena;
cellulose microbeads as described in patent application EP 1 562 562;
fibres, such as silk fibre, cotton fibre, wool fibre, flax fibre, cellulose fibre extracted especially from wood, from vegetables or from algae, polyamide fibre (Nylon®), modified cellulose fibre, poly-p-phenyleneterephthamide fibre, acrylic fibre, polyolefin fibre, glass fibre, silica fibre, aramid fibre, carbon fibre, Teflon® fibre, insoluble collagen fibre, polyester fibre, polyvinyl chloride or polyvinylidene chloride fibre, polyvinyl alcohol fibre, polyacrylonitrile fibre, chitosan fibre, polyurethane fibre, polyethylene phthalate fibre, fibres formed from a mixture of polymers, resorbable synthetic fibres, and mixtures thereof described in patent application EP 1 151 742;
expanded acrylic copolymer microspheres such as those sold by the company EXPANCEL under the name Expancel 551®;
fillers with an optical effect as described in patent application FR 2 869 796, in particular:
   polyamide powders (Nylon®), for instance Nylon 12 particles of the Orgasol type from Arkema, with a mean size of 10 microns and a refractive index of 1.54,
   silica powders, for instance Silica beads SB 150 from Miyoshi with a mean size of 5 microns and a refractive index of 1.45,
   polytetrafluoroethylene powders, for instance PTFE Ceridust 9205F from Clariant, with a mean size of 8 microns and a refractive index of 1.36,
   silicone resin powders, for instance the silicone resin Tospearl 145A from GE Silicone with a mean size of 4.5 microns and a refractive index of 1.41,
   acrylic copolymer powders, especially of polymethyl(meth)acrylate, for instance the PMMA particles Jurymer MBI from Nihon Junyoki, with a mean size of 8 microns and a refractive index of 1.49, or the Micropearl M100® and F 80 ED® particles from the company Matsumoto Yushi-Seiyaku,
   wax powders, for instance the paraffin wax particles Microease 114S from Micropowders, with a mean size of 7 microns and a refractive index of 1.54,
   polyethylene powders, especially comprising at least one ethylene/acrylic acid copolymer, and in particular consisting of ethylene/acrylic acid copolymers, for instance the particles Flobeads EA 209 from Sumitomo (with a mean size of 10 microns and a refractive index of 1.48),
   elastomeric crosslinked organopolysiloxane powders coated with silicone resin, especially with silsesquioxane resin, as described, for example, in patent U.S. Pat. No. 5,538,793. Such elastomeric powders are sold under the names KSP-100, KSP-101, KSP-102, KSP-103, KSP-104 and KSP-105 by the company Shin-Etsu, and
   talc/titanium dioxide/alumina/silica composite powders such as those sold under the name Coverleaf® AR-80 by the company Catalyst & Chemicals,
   mixtures thereof,
   compounds that absorb and/or adsorb sebum as described in patent application FR 2 869 796. Mention may be made especially of:
   silica powders, for instance the porous silica microspheres sold under the name Silica Beads SB-700 sold by the company Myoshi, the products Sunsphere® H51, Sunsphere® H33 and Sunsphere® H53 sold by the company Asahi Glass; the polydimethylsiloxane-coated amorphous silica microspheres sold under the name SA Sunsphere® H-33 and SA Sunsphere® H-53 sold by the company Asahi Glass;
   amorphous mixed silicate powders, especially of aluminium and magnesium, for instance the product sold under the name Neusilin UFL2 by the company Sumitomo;
   polyamide (Nylon®) powders, for instance Orgasol® 4000 sold by the company Arkema, and
   acrylic polymer powders, especially of polymethyl methacrylate, for instance Covabead® LH85 sold by the company Wackherr; of polymethyl methacrylate/ethylene glycol dimethacrylate, for instance Dow Corning 5640 Microsponge® Skin Oil Adsorber sold by the company Dow Corning, or Ganzpearl® GMP-0820 sold by the company Ganz Chemical; of polyallyl methacrylate/ethylene glycol dimethacrylate, for instance Poly-Pore® L200 or Poly-Pore® E200 sold by the company Amcol; of ethylene glycol dimethacrylate/lauryl methacrylate copolymer, for instance Polytrap® 6603 sold by the company Dow Corning;
   silicate particles, such as alumina silicate;
   mixed silicate particles, such as:
   magnesium aluminium silicate particles, such as saponite or hydrated magnesium aluminium silicate with a sodium sulfate sold under the trade name Sumecton® by the company Kunimine;
   the magnesium silicate, hydroxyethylcellulose, black cumin oil, marrow oil and phospholipids complex or Matipure® from Lucas Meyer, and
   mixtures thereof.

Preferred matting agents that may be used according to the invention include a pumpkin seed extract, a rice or corn starch, kaolinite, silicas, talc, polyamide powders, polyethylene powders, acrylic copolymer powders, expanded acrylic copolymer microspheres, silicone resin microbeads and mixed silicate particles, and mixtures thereof.

As active agent acting on keratinocyte proliferation that may be used in the composition according to the present invention, mention may be made of the extract of Larrea divaricata such as Capislow® from Sederma, mixtures of extracts of papaya, of olive leaves and of lemon, such as Xyléine® from Vincience, extract of hydrangea macrophylla leaf, for instance Amacha Liquid E® from Ichimaru Pharcos, retinol and esters thereof including retinyl palmitate, phloroglucinol, the nut cake extracts sold by Gattefosse and the Solanum tuberosum extracts such as Dermolectine® sold by Sederma.

The composition according to the present invention is suitable for topical application to the skin. The physiologically acceptable medium is preferentially a cosmetically or dermatologically acceptable medium, i.e. it has no odour, no unpleasant colour or appearance, and does not give rise to any unacceptable stinging, tautness or redness for the user.

The term “physiologically acceptable medium” means a medium that is compatible with human keratin materials, for instance the skin, mucous membranes, the nails, the scalp and/or the hair.

The composition according to the invention may be intended for cosmetic or pharmaceutical application, particularly dermatological application. Preferably, the composition according to the invention is intended for cosmetic application.

The composition may then comprise any constituent usually used in the intended application.

Mention may be made especially of water, solvents, oils of mineral, animal and/or plant origin, waxes, pigments, fillers, surfactants, cosmetic or dermatological active agents, UV-screening agents, polymers, gelling agents and preserving agents.

Needless to say, a person skilled in the art will take care to select this or these optional additional compound(s), and/or the amount thereof, such that the advantageous properties of the compounds according to the invention are not, or are not substantially, adversely affected by the envisaged addition.

The composition according to the invention may be in any galenical form normally used in cosmetics and dermatology; it may especially be in the form of an aqueous or aqueous-alcoholic solution, which is optionally gelled, a dispersion of the lotion type, which is optionally a two-phase dispersion, an oil-in-water or water-in-oil or multiple emulsion, an aqueous gel, a dispersion of oil in an aqueous phase with the aid of spherules, these spherules possibly being polymer nanoparticles such as nanospheres and nanocapsules or, better still, lipid vesicles of the ionic and/or non-ionic type, or alternatively a powder.

When the composition is an emulsion, the proportion of the fatty phase may range from 5% to 80% by weight and preferably from 5% to 50% by weight relative to the total weight of the composition. The oils, emulsifiers and possible co-emulsifiers used in the composition in emulsion form are chosen from those conventionally used in the field under consideration. The emulsifier and the co-emulsifier are present in the composition in a proportion that may range from 0.3% to 30% by weight and preferably from 0.5% to 20% by weight relative to the total weight of the composition.

This composition may be more or less fluid and may have the appearance of a white or coloured cream, an ointment, a milk, a lotion, a serum, a paste or a mousse. It may optionally be applied to the skin in aerosol form. It may also be in solid form, for example in stick form.

The examples that follow illustrate the present invention.

### EXAMPLE 1

Measurement of the melanogenesis-modulating effect of the copolymer of styrene/hydrolysed maleic anhydride (50/50) in sodium salt form, at 40% in water (SMA1000HNa® from the company Cray Valley), in the presence or absence of ascorbic acid, on a coculture of normal human keratinocytes and melanocytes.

1.1. Materials and Method

The melanogenesis-modulating effect of the copolymer SMA1000HNa® was tested according to the method described in patent FR-A-2 734 825, and also in the article by R. Schmidt, P. Krien and M. Régnier, Anal. Biochem., 235(2), 113-18, 1996.

Briefly, the method comprises the steps consisting in:

coculturing normal human keratinocytes/melanocytes in a medium containing the tested extract and also a labelled melanin precursor, in the present case C14-labelled thiouracil,

lysing the cells containing the melanin with protein-degrading enzymes,

passing the extract obtained through an anion-exchange filter with a pore diameter of less than or equal to 1 μm, and

evaluating the amount of melanin bound by the filter by means of radioactive assay of the thiouracil.

Specifically, 50 000 normal human keratinocytes and 17 000 normal human melanocytes are mixed together and inoculated per well in 96-well plates (Costar type) and cultured for three days in the differentiation medium. During the following three days, the culture medium is replaced daily with the defined test medium (containing the products to be evaluated, and also 3 μCi/ml of C14-labelled thiouracil).

The following controls are prepared:
control culture: test medium
positive melanogenesis-stimulating control: test medium+1 mM tyrosine;
   3 positive melanogenesis-inhibiting controls: test medium+, respectively, 500 μM of kojic acid; 100 μM of arbutin; 10% M of lucinol (2-butyl-1,3-benzenediol).

The total radioactivity incorporated into the proteins is estimated by the incorporation of tritiated leucine, taken as the proliferation indicator and the early cytotoxicity indicator. On the day before the sample is taken, 1 μCi/ml of tritiated leucine is added to the test medium. It is considered that a product is cytotoxic when the content of tritiated leucine falls by at least 30%.

After one night, the cells are rinsed in phosphate buffer (phosphate-buffered saline, PBS). The proteins are precipitated using 5% trichloracetic acid (TCA) and washed in order to remove the free radioactivity. The proteins are incubated overnight at 37° C. using a proteinase K solution at 20 μl/ml in tris-HCl-Triton EDTA buffer.

50 μl of total extract are taken and transferred into a 96-well plate (Wallac) and 150 μl of scintillation liquid (Optiphase “Supermix”) are added. The rest of the extract, i.e. 950 μl, is filtered through a DEAE Filtermat filter. After rinsing, the filter covered with the “Meltilex” solid scintillant is transferred into a counting plate. The radioactivity is counted using a Wallac counter. The results are expressed as a percentage of the control according to the formula:

$$\frac{( 14 CP / 3 HP ) - ( 14 CT / 3 HT )}{( 14 CT / 3 HT )} 100$$

in which:

14CP is the mean of the disintegrations per minute (dpm) of 14C-thiouracil on 3 similar wells treated with a product (P);

3HP is the mean of the dpm of the corresponding 3H-leucine;

14CT is the mean of the dpm of 14 C-thiouracil on 3 similar control wells (T);

3HT is the mean of the dpm of the corresponding 3H-leucine.

The ratio of the incorporation of thiouracil to the incorporation of leucine is calculated, to normalize the melanogenesis to the amount of proteins present in the well. This ratio reflects the melanogenesis modulation.

1.2. Results

| | ³H Leu. | ¹⁴C ThioU. | ¹⁴C ThioU. |
|---|---|---|---|
| References | (%/Control) | (%/Control) | ³H Leu. |
| Tyrosine 1 mM | +6% | 96% | 85% |
| Kojic acid 500 μM | −8% | −56% | −52% |
| Arbutin 100 μM | −8% | −49% | −44% |
| Lucinol 10 μM | 4% | −60% | −62% |

| | | | | | |
|---|---|---|---|---|---|
| [vitamin C] | 0.1 | 0.3 | 0.4 | 0.6 | IC50 |
| (mM) | | | | | |
| ³H Leu. | 0% | −17% | −23% | −23% | |
| (%/Control) | | | | | |
| ¹⁴C ThioU. | −14% | −69% | −79% | −76% | |
| (%/Control) | | | | | |
| ¹⁴C ThioU. | −14% | −62% | −72% | −68% | 0.23 mM |
| ³H Leu. | | | | | vitamin C |
| [vitamin C] | 0.1 | 0.3 | 0.4 | 0.6 | |
| (mM) | | | | | |
| [copolymer SMA] | 18 | 53 | 70 | 105 | IC50 |
| (μM) | | | | | |
| ³H Leu. | −4% | −15% | −12% | −11% | |
| (%/Control) | | | | | |
| ¹⁴C ThioU. | −28% | −66% | −67% | −71% | |
| (%/Control) | | | | | |
| ¹⁴C ThioU. | −25% | −60% | −63% | −68% | 0.23 mM vitamin C + |
| ³H Leu. | | | | | 42 μM SMA |
| [copolymer SMA] | 18 | 53 | 70 | 105 | IC50 |
| (μM) | | | | | |
| ³H Leu. | 2% | 4% | 0% | −16% | |
| (%/Control) | | | | | |
| ¹⁴C ThioU. | −21% | −42% | −56% | −62% | |
| (%/Control) | | | | | |
| ¹⁴C ThioU. | −23% | −45% | −56% | −54% | 60 μM SMA |
| ³H Leu. | | | | | |

In the tables, SMA means the copolymer SMA1000HNa ®

1.3. Conclusions

No cytotoxicity of the tested products was observed at the evaluated doses. Vitamin C has depigmenting activity and achieves the IC50 on this model at about 0.23 mM. The activity of vitamin C is not modified in the presence of the copolymer SMA1000HNa®. On the other hand, the copolymer SMA1000HNa® tested alone, over the same concentration range as in the presence of vitamin C, also has a depigmenting potential with a dose effect. It reaches the IC50 at 60 μM of copolymer. The copolymer SMA1000HNa® thus has intrinsic depigmenting activity.

The activities of vitamin C and of the copolymer SMA1000HNa® are not synergistic. The activity of the copolymer SMA1000HNa® appears, on the contrary, to be masked in the presence of vitamin C.

### EXAMPLE 2

### 2) Principle of the Anti-Pigmentation Protocol

Measurement of the photoinduced melanogenesis-modulating effect of the polymer SMA1000HNa® in the presence or absence of ascorbic acid on a coculture of normal human keratinocytes and melanocytes (anti-pigmentation effect).

2.1. Materials and Method

Normal human melanocyte/keratinocyte cocultures (NHM/NHK) undergo 1 irradiation with UV radiation, repeated daily for 4 days, in the presence or absence of the products to be evaluated. The melanin synthesis is quantified as previously by means of the incorporation of ¹⁴C-thiouracil present in the culture medium. In parallel, a protein assay is performed in each well in order to normalize the melanin synthesis and to estimate the toxicity of the product. Attenuation of the melanogenesis induction after UV irradiation may thus be demonstrated.

More specifically, the melanocytes and keratinocytes are inoculated, respectively, at 80 000 and 250 000 cells per well in 24-well plates in the keratinocyte differentiation medium. The plates are incubated for 3 days at 37° C. before treatment.

To avoid photo-oxidation of one of its components, the culture medium is replaced at the time of irradiation with a solution of the products in PBS. The products are placed in contact with the cells for 15 minutes before irradiation.

The UV irradiation is performed using a sun simulator. The delivered dose is 7 J/cm². Irradiation source: Oriel Xénon 1000W simulator+dichroic filter+WG 320/1.5 mm.

The following controls are prepared:

Non-irradiated control (quantification of the constitutive melanogenesis)

Blank irradiation control

2 positive controls of photoinduced melanogenesis inhibition: (100 μM arbutin; 1 μM lucinol).

After irradiation, the PBS is removed and the cells are incubated in a semi-defined culture medium to which are added the products and the ¹⁴C-thiouracil (1 μCi/ml). This treatment is repeated daily for 4 days.

For each experimental condition, cell lysates are performed by rinsing the cells with a phosphate buffer (PBS). The proteins are precipitated using 5% trichloracetic acid (TCA) and washed in order to remove the free radioactivity. The proteins are incubated over a weekend at 37° C. using a proteinase K solution at 20 μl/ml in tris HCl-Triton-EDTA buffer.

5 μl of total extract are taken and transferred into a 96-well plate (Wallac). The proteins are assayed using the Protein Assay Reagent assay kit (Pierce). The amount of proteins per well (P) will be used to normalize the melanogenesis. The rest of the extract is filtered on a DEAE Filtermat filter (Wallac). After rinsing, the filter covered with “Meltilex” solid scintillant (Wallac) is transferred into a Costar counting plate. The radioactivity is counted using a Wallac counter and allows quantification of the melanogenesis (M) by measurement of the ¹⁴C-thiouracil (ccpm) incorporated by the cells.

Normalized melanogenesis (NM)=M/P

Photo-induced melanogenesis (PIM)=NM (irradiated sample)−NM (non-irradiated sample)

Anti-pigmenting effect of the product (%)=[[PIM(irradiated+treated sample)−PIM(irradiated sample)]/PIM(irradiated sample)]×100

2-2 Results

| | Proteins | PIM | Anti- |
|---|---|---|---|
| | (%/non- | (%/non- | pigmenting |
| | irradiated | irradiated | effect of the |
| References | control) | control) | product |
| Control SSR | −21% | 225% | — |
| Arbutin 100 μM + SSR | −15% | −24% | −111% |
| Lucinol 1 μM + SSR | −14% | −36% | −116% |

| | [vitamin C] (mM) | | | | |
|---|---|---|---|---|---|
| | 0.3 | 1 | 2 | 3 | IC50 |
| Proteins | −12% | −19% | −19% | −22% | |
| (%/non-irradiated | | | | | |
| control) | | | | | |
| PIM | 134% | 78% | 23% | 0% | |
| (%/non-irradiated | | | | | |
| control) | | | | | |
| Anti-pigmenting | −40% | −65% | −90% | −100% | 0.47 mM |
| effect of the | | | | | vitamin C |
| product | | | | | |

| | [vitamin C] (mM) | | | | |
|---|---|---|---|---|---|
| | 0.3 | 1 | 2 | 3 | |
| | [SMA] (μM) | | | | |
| | 53 | 180 | 350 | 530 | IC50 |
| Proteins | −24% | −26% | −25% | −27% | |
| (%/non-irradiated | | | | | |
| control) | | | | | |
| PIM | 118% | 69% | 17% | −5% | |
| (%/non-irradiated | | | | | |
| control) | | | | | |
| Anti-pigmenting | −47% | −69% | −92% | −102% | 0.37 mM vitamin C + |
| effect of the | | | | | 67 μM SMA |
| product | | | | | |

| | [SMA] (μM) | | | | |
|---|---|---|---|---|---|
| | 53 | 180 | 350 | 530 | IC50 |
| Proteins | −24% | −23% | −22% | −20% | |
| (%/non-irradiated | | | | | |
| control) | | | | | |
| PIM | 189% | 126% | 160% | 166% | |
| (%/non-irradiated | | | | | |
| control) | | | | | |
| Anti-pigmenting | — | −44% | −29% | −26% | Not reached |
| effect of the | | | | | |
| product | | | | | |

2.3. Conclusions

No cytotoxicity of the test products was observed at the evaluated doses. Vitamin C has anti-pigmenting activity and reaches the IC50 on this model at about 0.47 mM. The activity of vitamin C at low concentrations under UV is substantially improved in the presence of the copolymer SMA1000HNa®. Specifically, the IC50 of vitamin C is reached at 0.37 mM in the presence of the copolymer SMA1000HNa®, i.e. more quickly than when it is tested alone. This slight improvement might be due to the stabilizing potential of the copolymer SMA1000HNa® on vitamin C, under these pro-oxidizing experimental conditions of exposure to UV rays. Nevertheless, the copolymer SMA1000HNa® tested alone, over the same concentration range as in the presence of vitamin C, also has an intrinsic anti-pigmenting potential. This effect is not synergistic with that of vitamin C.

### EXAMPLE 3

### Shaving Foam

| Ingredients | weight % |
|---|---|
| Fragrance | 0.60 |
| Triethanolamine | 2.90 |
| Butane | 1.00 |
| 2,4,4′-Trichloro-2′-hydroxydiphenyl ether | 0.10 |
| Dimethiconol stearate (Mirasil wax S from Rhodia) | 1.00 |
| Potassium hydroxide | 0.40 |
| Oxypropylated (2 PO) coconut acid monoethanolamide | 1.00 |
| (Promidium CO from Uniqema) | |
| Polyethylene glycol (14 000 EO) | 0.50 |
| Sodium laureth sulfate | 1.00 |
| Myristic acid | 0.20 |
| Glycerol | 5.00 |
| Palmitic acid | 3.80 |
| Stearic acid | 4.60 |
| Pyridoxine hydrochloride (vitamin B6)/anti-seborrhoeic | 0.04 |
| active agent | |
| Isobutane | 2.38 |
| Propane | 0.85 |
| Hydroxyethylcellulose | 0.28 |
| Preserving agent | 0.95 |
| Copolymer of styrene/maleic acid, sodium salt, | 0.50 |
| at 40% by weight in water * | |
| Mint leaf extract in aqueous solution (Calmiskin ® | 0.50 |
| from Silab) calmative | |
| Tamanu oil cicatrizing agent | 0.10 |
| Water | qs 100 |

* SMA1000HNa ® sold by the company Cray Valley

This shaving foam allows seborrhoea to be treated and prevents the appearance of hyperpigmentation that may occur on acne-prone black skin.

### Example 4: medicated cream

| Ingredients | Weight % |
|---|---|
| Polystearyl acrylate (Intelimer IPA 13-1 from Landec) | 1.30 |
| Magnesium sulfate | 0.70 |
| Preserving agent | 0.80 |
| Zinc pidolate/anti-seborrhoeic active agent | 0.25 |
| Vinylidene chloride/acrylonitrile/polymethyl | 0.50 |
| methacrylate microspheres expanded with isobutane | |
| (Expancel 551 from Expancel) | |
| Copolymer of styrene/maleic acid, sodium salt, at | 1.00 |
| 40% by weight in water * | |
| Mixture of acetylated ethylene glycol stearate and | 0.50 |
| glyceryl tristearate (Unitwix from United Guardian) | |
| Isohexadecane | 7.75 |
| Cyclohexadecane | 8.70 |
| Glycerol | 7.00 |
| Rose petal extract (Rose Flower Herbasol ® extract | 1.00 |
| from Cosmetochem) anti-irritant | |
| Isononyl isononanoate | 7.75 |
| Disodium salt of diethylenetetracetic acid | 0.04 |
| Ethyl dimethicone copolyol (Abil EM 90 from | 3.80 |
| Goldschmidt) | |
| Octadecene dioic acid desquamating agent | 0.50 |
| Polyglyceryl-4 isostearate (Isolan GI 34 from | 1.25 |
| Goldschmidt) | |
| Water | qs 100 |

* SMA1000HNa ® sold by the company Cray Valley

This cream applied daily in the evening is used for treating acne-prone greasy skin and prevents the hyperpigmentation that may occur on black skin.

### EXAMPLE 5

### Anti-Acne Medicated Lotion

| Ingredients | Weight % |
|---|---|
| Tocopherol | 0.05 |
| Myristyltrimethylammonium bromide | 0.03 |
| Disodium salt of diethylenetetracetic acid | 0.08 |
| Polyethylene glycol (8 EO) | 5.0 |
| Glycerol | 3.0 |
| Oxyethylenated (60 EO) hydrogenated castor oil | 0.5 |
| Copolymer of styrene/maleic acid, sodium salt, | 0.5 |
| at 40% by weight in water * | |
| Salicylic acid desquamating agent | 0.5 |
| Preserving agents | 0.8 |
| Fragrance | 0.08 |
| Caprylyl glycol anti-acne agent | 0.2 |
| Rose petal extract (Rose Flower Herbasol ® | 1.0 |
| extract from Cosmetochem) anti-irritant agent | |
| Glycine grafted onto an undecylenic chain (Lipacide | 0.5 |
| UG OR from SEPPIC) anti-seborrhoeic agent | |
| Ethanol | 5.0 |
| Water | qs 100 |

* SMA1000HNa ® sold by the company Cray Valley

The lotion is applied to acne spots on black skin once a day, in the evening. It allows the spots to disappear without forming hyperpigmentation marks.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Composition comprising, in a physiologically acceptable medium, at least one copolymer of a styrene monomer and of an ethylenically unsaturated dicarboxylic acid and at least one anti-seborrhoeic active agent.

2. Composition according to claim 1, in which the styrene monomer is chosen from styrene and α-methylstryene.

3. Composition according to claim 1, in which the styrene monomer is styrene.

4. Composition according to claim 1, in which the ethylenically unsaturated dicarboxylic acid is chosen from maleic acid and itaconic acid, and anhydride derivatives thereof hydrolysed after polymerization.

5. Composition according to claim 1, in which the ethylenically unsaturated dicarboxylic acid is chosen from maleic acid or an anhydride derivative thereof hydrolysed after polymerization.

6. Composition according to claim 1, in which the said copolymer is in salt form.

7. Composition according to claim 6, in which the said copolymer is in the form of a salt chosen from the ammonium, sodium, potassium and lithium salts.

8. Composition according to claim 1, in which the said copolymer is a copolymer of styrene and of maleic acid.

9. Composition according to claim 1, in which the anti-seborrhoeic active agent is chosen from retinoic acid; benzoyl peroxide; sulfur; vitamin B6; selenium chloride; sea fennel; mixtures of extract of cinnamon, of tea and of octanoylglycine; the mixture of cinnamon, sarcosine and octanoylglycine; zinc salts; copper derivatives; extracts of plants of the species Arnica montana, Cinchona succirubra, Eugenia caryophyllata, Humulus lupulus, Hypericum perforatum, Mentha piperita, Rosmarinus officinalis, Salvia oficinalis and Thymus vulgaris; extracts of meadowsweet; extracts of the alga Laminaria saccharina; mixtures of extracts of salad burnet root, of ginger rhizome and of cinnamon bark; linseed extracts; Phellodendron extracts; mixtures of argan oil, of Serenoa serrulata extract and of sesame seed extract; mixtures of extracts of willowherb, of Terminalia chebula, of nasturtium and of bioavailable zinc; extracts of Pygeum afrianum; extracts of Serenoa serrulata; mixtures of extracts of plantain, of Berberis aquifolium and of sodium salicylate; clove extract; argan oil; lactic protein filtrates; extracts of the alga Laminaria; oligosaccharides of the alga Laminaria digitata; sugar cane extracts; sulfonated shale oil; European meadowsweet extracts; sebacic acid; glucomannans extracted from konjac tuber and modified with alkylsulfonate chains; extracts of Sophora angustifolia; extracts of Cinchona succirubra bark; extracts of Quillaja saponaria; glycine grafted onto an undecylenic chain; the mixture of oleanolic acid and of nordihydroguaiaretic acid; phthalimidoperoxyhexanoic acid; tri(C₁₂-C₁₃)alkyl citrate; tri(C₁₄-C₁₅)alkyl citrate; 10-hydroxydecanoic acid; mixtures of 10-hydroxydecanoic acid, of sebacic acid and of 1,10-decanediol, and mixtures thereof.

10. Composition according to claim 1, in which the anti-seborrhoeic active agent is chosen from benzoyl peroxide; vitamin B6; zinc salts; meadowsweet extracts; extracts of the alga Laminaria saccharina; mixtures of extracts of salad burnet root, of ginger rhizome and of cinnamon bark; clove extract; lactic protein filtrates; European meadowsweet extracts; sebacic acid; glycine grafted onto an undecylenic chain; tri(C₁₂-C₁₃)alkyl citrate; tri(C₁₄-C₁₅)alkyl citrate; 10-hydroxydecanoic acid; mixtures of 10-hydroxydecanoic acid, of sebacic acid and of 1,10-decanediol, and mixtures thereof.

11. Composition according to claim 1, in which the anti-seborrhoeic active agent is chosen from zinc salts; clove extract; glycine grafted onto an undecylenic chain; tri(C₁₂-C₁₃)alkyl citrate; tri(C₁₄-C₁₅)alkyl citrate, and mixtures thereof.

12. Composition according to claim 1, in which the anti-seborrhoeic active agent is present in a content ranging from 0.1% to 10% by weight relative to the total weight of the composition.

13. Composition according to claim 1, in which an anti-acne active agent is also present.

14. Composition according to claim 13, in which the anti-acne active agent is a bactericide optionally supplemented with an active agent with bacterial anti-adhesion effects or an agent that acts on the biofilm of bacteria to prevent them from proliferating.

15. Composition according to claim 14, in which the bactericide is chosen from Asiatic acid, the monoethanolamine salt of 1-hydroxy-4-methyl 6-trimethylpentyl-2-pyridone; citronellic acid, perillic acid; glyceryl 2-ethylhexyl ether; glyceryl caprylate/caprate; sodium calcium phosphosilicate; silver-based particles; hop cone extract; St.-John's Wort extract; the mixture of extracts of roots of Scutellaria baicalensis, of Paeonia suffruticosa and Glycyrrhiza glabra; argan tree extract; bearberry leaf extracts; 10-hydroxy-2-decanoic acid, sodium ursolate, azelaic acid, diiodomethyl p-tolyl sulfone, malachite powder, zinc oxide, octadecenedioic acid; ellagic acid; 2,4,4′-trichloro-2′-hydroxydiphenyl ether, 1-(3′,4′-dichlorophenyl)-3-(4′-chlorophenyl)urea, 3,4,4′-trichlorocarbanilide, 3′,4′,5′-trichlorosalicylanilide, phenoxyethanol, phenoxypropanol, phenoxyisopropanol, hexamidine isethionate, metronidazole and salts thereof, miconazole and salts thereof, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and salts thereof, benzoyl peroxide, 3-hydroxybenzoic acid, 4-hydroxybenzoic acid, phytic acid, N-acetyl-L-cysteine, lipoic acid, azelaic acid and salts thereof, arachidonic acid, resorcinol, 3,4,4′-trichlorocarbanalide, octoxyglycerine, octanoylglycine, caprylyl glycol, 10-hydroxy-2-decanoic acid, dichlorophenylimidazoldioxolane and derivatives thereof, iodopropynyl butylcarbamate, 3,7,11-trimethyldodeca-2,5,10-trienol, phytosphingosines; quaternary ammonium salts, cetylpyridinium salts, sodium cocoamphoacetate, disodium diacetate, betaines, sodium lauryl ether sulfate, decyl glucoside, branched C₁₂₋₁₃ dialkyl malates, propylene glycol monoesters, lauryldimethylamine betaine, polyquaternary ammoniums, and mixtures thereof.

16. Composition according to the claim 14, in which the active agent with bacterial anti-adhesive effects is chosen from phytanetriol and derivatives thereof; plant oils such as wheatgerm oil, calendula oil, castor oil, olive oil, avocado oil, sweet almond oil, groundnut oil, jojoba oil, sesame oil, apricot kernel oil, sunflower oil or macadamia oil; disodium cocoamphodiacetate, oxyethylenated (7 EO) glyceryl cocoate, 18-hexadecenyl succinate, octoxyglyceryl palmitate, octoxyglyceryl behenate, dioctyl adipate, PPG-15 stearyl ether, branched C₁₂-C₁₃ dialkyl tartrates, and mixtures thereof.

17. Composition according to claim 16, in which the active agent that acts on the biofilm of bacteria to prevent them from proliferating is chosen from pentylene glycol, Nylon-66; rice bran oil; polyvinyl alcohol; rapeseed oil; fructose derivatives, and mixtures thereof.

18. Composition according to claim 13, in which the anti-acne active agent is present in a content ranging from 0.01% to 10% by weight relative to the total weight of the composition.

19. Composition according to claim 1, in which a desquamating agent is also present.

20. Composition according to claim 19, in which the desquamating agent is chosen from β-hydroxy acids; α-hydroxy acids; 8-hexadecene-1,16-dicarboxylic acid; gentisic acid and derivatives thereof; oligofucoses; cinnamic acid; Saphora japonica extract; resveratrol and certain jasmonic acid derivatives; aminosulfonic compounds; 2-oxothiazolidine-4-carboxylic acid derivatives; derivatives of α-amino acids of glycine type; honey; O-octanoyl-6-D-maltose and N-acetylglucosamine; oligofructoses, EDTA and derivatives thereof, laminaria extracts, o-linoleyl-6D-glucose, (3-hydroxy-2 pentylcyclopentyl)acetic acid, glycerol trilactate, O-octanyl-6′-D-maltose, S-carboxymethylcysteine, siliceous derivatives of salicylate, oligofucases, jasmonic acid and derivatives thereof; extract of the flowers of ficus Opuntia indica, and mixtures thereof.

21. Composition according to claim 1, in which at least one calmative and/or at least one astringent active agent and/or at least one matting active agent and/or at least one cicatrizing active agent and/or at least one active agent acting on keratinocyte proliferation is also present.

22. Cosmetic process for treating greasy skin and/or hyperseborrhoea and for preventing the acne lesions resulting therefrom and/or for combating aesthetic disorders or imperfections associated with the overproduction of sebum on dark skin types, comprising at least one step that consists in applying to the skin a composition as defined according to claim 1.

23. Cosmetic process for combating both greasy skin and hyperpigmentation localized at the sites of cicatrization of acne lesions resulting from the hyperseborrhoea of dark skin types, comprising at least one step that consists in applying to the skin a composition as defined according to claim 1.

24. A dermatological method for treating greasy skin and/or the hyperseborrhoea of dark skin types comprising the topical application to zones of the skin of a person in need thereof of an effective amount of a composition comprising a combination of at least one copolymer of a styrene monomer and of an ethylenically unsaturated dicarboxylic acid and of at least one anti-seborrhoeic active agent.

25. Composition according to claim 1, in which the said copolymer is in the form of a sodium salt.
